(19) United States
(12) Patent Application Publication
(10) Pub. No.: US 2009/0068683 A1
(43) Pub. Date: Mar. 12, 2009

(54) MARKERS FOR PREECLAMPSIA

(75) Inventor: Vesna D. Garovic, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research, Rochester, MN (US)

(21) Appl. No.: 12/137,350

(22) Filed: Jun. 11, 2008

Related U.S. Application Data
(60) Provisional application No. 60/943,242, filed on Jun. 11, 2007.

(51) Int. Cl. C12Q 1/02; G01N 33/53

(52) U.S. Cl. 435/7.21; 435/29

(57) ABSTRACT

This document provides methods and materials related to determining whether or not a pregnant mammal (e.g., a pregnant human) has preeclampsia. For example, methods and materials related to the use of urinary podocytes to determine whether or not a pregnant human has preeclampsia are provided.

### CROSS-REFERENCE TO RELATED APPLICATIONS

This application claims the benefit of U.S. Provisional Application Ser. No. 60/943,242, filed Jun. 11, 2007. The disclosure of the prior application is incorporated by reference in its entirety.

### BACKGROUND

1. Technical Field

This document relates to methods and materials involved in determining whether or not a pregnant mammal has preeclampsia. For example, this document provides methods and materials related to the use of urinary podocytes to determine whether or not a pregnant mammal (e.g., a pregnant human) has preeclampsia.

2. Background Information

Preeclampsia is a pregnancy-specific disease that affects about 5 percent of all pregnancies and remains a leading cause of both maternal and fetal morbidity and death worldwide. It is characterized by hypertension (blood pressure, ≧140/90 mm Hg) and proteinuria (≧300 mg in a 24-hour urine sample) that occur after 20 weeks of gestation. Proteinuria in preeclampsia is associated with characteristic renal pathologic changes of glomerular endotheliosis, which is considered to be a hallmark of preeclampsia in humans.

### SUMMARY

This document provides methods and materials related to determining whether or not a pregnant mammal (e.g., a pregnant human) has preeclampsia. For example, this document provides methods and materials related to the use of urinary podocytes to determine whether or not a pregnant human has preeclampsia. Identifying patients who have preeclampsia can allow such patients, who are at risk for both maternal and fetal morbidity and death, to be treated effectively. In addition, identifying patients who do not have preeclampsia can avoid unnecessary treatment and patient suffering. As described herein, the presence of urinary podocytes can be used to identify pregnant humans as having preeclampsia.

In general, one aspect of this document features a method for assessing a pregnant mammal for preeclampsia. The method comprises, or consists essentially of, determining whether or not a urine sample from the mammal contains an elevated level of urinary podocytes, wherein the presence of the elevated level indicates that the mammal has preeclampsia. The mammal can be a human. The determining step can comprise using an antibody to detect podocytes. The antibody can be an anti-podocin antibody. The antibody can be an anti-podocalyxin antibody. The antibody can be an anti-nephrin antibody. The antibody can be an anti-synaptopodin antibody. The method can comprise classifying the mammal as having preeclampsia if the elevated level is present, and classifying the mammal as not having preeclampsia if the elevated level is not present.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains photographs of urinary cells plated on collagen-coated slides, cultured for 24 hours, and stained for podocin (A), podocalyxin (B), nephrin (C), and synaptopodin (D) immunoreactivity.

FIG. 2 contains graphs plotting ROC curves for podocyturea as determined by staining for podocin (A), podocalyxin (B), nephrin (C), and synaptopodin (D) immunoreactivity.

FIG. 3 contains graphs plotting sFlt-1 (A; pg/mL), soluble endoglin (B; ng/mL), serum PIGF (C; pg/mL), and urine PIGF (D; adjusted for Mg creatinine) for normotensive and preeclamptic pregnancies as a function of age. For normotensive pregnancies, open circles indicate individual values with a trend regression line. The dashed portion represents extrapolation. For preeclamptic pregnancies, values are stratified by intervals of gestational age in which data distribution are summarized with box plots indicating median values and interquartile ranges.

FIG. 4 contains graphs plotting ROC curves for sFlt-1 (A), soluble endoglin (B), serum PIGF (C), and urine PIGF (D).

### DETAILED DESCRIPTION

This document provides methods and materials related to determining whether or not a pregnant mammal (e.g., a pregnant human) has preeclampsia. For example, this document provides methods and materials related to the use of urinary podocytes to determine whether or not a pregnant human has preeclampsia. As disclosed herein, if the level of urinary podocytes is elevated in a pregnant mammal, then the mammal can be classified as having preeclampsia. If the level of urinary podocytes is not elevated in a pregnant mammal, then the mammal can be classified as not having preeclampsia.

The methods and materials provided herein can be used to assess any pregnant mammal for preeclampsia. For example, a human, cat, dog, or horse can be assessed for preeclampsia. In some cases, a human pregnant for 18 to 36 weeks (e.g., between 18 and 35 weeks, between 20 and 35 weeks, or between 20 and 30 weeks) can be assessed.

Any appropriate method can be used to determine the level of podocytes in a mammal's urine. For example, cell staining techniques that include using antibodies that bind to podocytes or polypeptides expressed by podocytes can be used. Examples of such antibodies include, without limitation, antibodies that have the ability to bind podocin, podocalyxin, nephrin, synaptopodin, Neph1, GLEPP1, WT1, CD2AP, actin, actinin, cadherin, catenin, integrin, vinculin, talin, paxillin, and ZO-1.

The term “elevated level” as used herein with respect to the level of urinary podocytes is any level that is above a median urinary podocytes level in urine from a random population of pregnant mammals (e.g., a random population of 10, 20, 30, 40, 50, 100, or 500 pregnant mammals) lacking preeclampsia. In some cases, an elevated level of urinary podocytes can be a detectable level of podocin-positive cells within a urine sample. The presence or absence of such a detectable level of podocin-positive cells can be determined using an anti-podocin antibody.

An antibody can be, without limitation, a polyclonal, monoclonal, human, humanized, chimeric, or single-chain antibody, or an antibody fragment having binding activity, such as a Fab fragment, F(ab′) fragment, Fd fragment, fragment produced by a Fab expression library, fragment comprising a VL or VH domain, or epitope binding fragment of any of the above. An antibody can be of any type (e.g., IgG, IgM, IgD, IgA or IgY), class (e.g., IgG1, IgG4, or IgA2), or subclass. In addition, an antibody can be from any animal including birds and mammals. For example, an antibody can be a human, rabbit, sheep, or goat antibody. An antibody can be naturally occurring, recombinant, or synthetic. Antibodies can be generated and purified using any suitable methods known in the art. For example, monoclonal antibodies can be prepared using hybridoma, recombinant, or phage display technology, or a combination of such techniques. In some cases, antibody fragments can be produced synthetically or recombinantly from a gene encoding the partial antibody sequence. An anti-podocin antibody can bind to podocin polypeptides at an affinity of at least 10⁴ mol⁻¹ (e.g., at least 10¹, 10⁶, 10⁷, 10⁸, 10⁹, 10¹⁰, 10¹¹, or 10¹² mol⁻¹).

Once the level of urinary podocytes of a mammal is determined, then the level can be compared to a median level or a cutoff level and used to evaluate the mammal for preeclampsia. A level of urinary podocytes that is higher than the median level of urinary podocytes from a population of mammals of the same species having no preeclampsia (or a cutoff level) can indicate that the mammal has preeclampsia. A level of urinary podocytes that is lower than the median level of urinary podocytes from a population of mammals of the same species having no preeclampsia (or a cutoff level) can indicate that the mammal does not have preeclampsia. A cutoff level can be set to any level provided that the values greater than that level correlate with an increased level of urinary podocytes indicative of a mammal having preeclampsia. For example, a cutoff level can be equal to, or greater than 1 cell/mg creatinine. In some cases, a pregnant mammal can be classified as having preeclampsia if it is determined that the podocyte level in a urine sample from the mammal is greater than the podocyte level in a urine sample obtained previously from that mammal.

A mammal that has been or is being treated for preeclampsia can be monitored using the methods and materials provided herein. For example, the level of podocytes in a urine sample from a mammal being treated for preeclampsia can be assessed to determine whether or not the mammal is responding to the treatment.

This document also provides methods and materials to assist medical or research professionals in determining whether or not a pregnant mammal has preeclampsia. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of urinary podocytes in a urine sample, and (2) communicating information about the level to that professional.

Any appropriate method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

The invention will be further described in the following examples, which do not limit the scope of the invention described in the claims.

### EXAMPLES

### Example 1

### Urinary Podocyte Excretion as a Marker for Preeclampsia

An approved study was conducted with the consent of all included women. A diagnosis of preeclampsia was made in the presence of (a) hypertension after 20 weeks of gestation, which was defined as a blood pressure of ≧140/90 mm Hg, (b) proteinuria, which was defined as ≧300 mg of protein in a 24-hour urine specimen, and/or 1+(30 mg/L) dipstick urinalysis in the absence of urinary tract infection and/or a predicted 24-hour urine protein of ≧300 mg on a random urine collection, and (c) resolution of hypertension and proteinuria by 12 weeks after delivery. Women with severe forms of preeclampsia such as eclampsia and HELLP (hemolysis, elevated liver enzymes, low platelets) syndrome, the diagnosis of which was confirmed on the basis of previously published criteria (Jones, Hematopathol. Mol. Hematol., 11:147-71 (1998)), also were included. Healthy, normotensive pregnant women without hypertension and proteinuria served as control subjects. An additional control group consisted of women with hypertension and proteinuria.

A cross-sectional study was conducted, and blood and urine samples were collected close to and typically ≦24 hours before delivery. In total, 67 women were recruited. Preeclampsia was present in 33 of the patients, and HELLP was diagnosed in 11 patients; 23 normotensive pregnancies served as control subjects (Table 1). Blood samples were obtained in all 67 women, and urine samples for podocyturia were collected in a subset of 31 pregnant women (15 cases and 16 control subjects).

TABLE 1
Patient characteristics.

| | | | | Preeclampsia + |
|---|---|---|---|---|
| Variable | Normal (n = 23) | Preeclampsia (n = 33) | HELLP (n = 11) | HELLP (n = 44) |
| Maternal age (y) | 28.7 ± 5.4 | 26.2 ± 5.1 | 33.0 ± 6.0 | 27.9 ± 6.1 |
| Gestational age (wk) | 39.2 ± 2.2 | 34.3 ± 3.8* | 33.5 ± 5.6* | 34.1 ± 4.2* |
| Primiparous (%) | 47.8 | 81.8 | 9.1 | 63.6 |
| Systolic blood | 110.5 ± 9.5 | 159 ± 19.8* | 162.6 ± 23* | 159.9 ± 20.3* |
| pressure (mm Hg) | | | | |
| Diastolic blood | 66.9 ± 9.8 | 97.8 ± 9.4* | 98.3 ± 10.6* | 97.9 ± 9.6* |
| pressure (mm Hg) | | | | |
| Proteinuria (g/24 hr) | 247 ± 294 | 2693 ± 3164* | 4373 ± 5962* | 3113 ± 4032* |
| Platelet count | 242,000 ± 35,519 | 232,333 ± 66,787 | 100,273 ± 42,245* | 199,318 ± 84,146 |

Data are given as mean ± SD.
*P < .05, compared with normal group.

### Serum Studies

Blood samples for the determination of sFlt-1, free PlGF, and soluble endoglin levels were drawn within 24 hours before delivery. Serum creatinine level, liver function tests, and platelet counts were performed according to standardized laboratory procedures. Serum levels of sFlt-1, soluble endoglin, and free PlGF were measured with Quantikine ELISA (enzyme-linked immunosorbent assay) kits (R&D Systems, Minneapolis, Minn.).

### Urine Chemistry

Concurrent with serum collection, clean-catch urine specimens (50-100 mL) were obtained. Urine albumin, total protein, and creatinine concentrations were measured by standard methods on a Hitachi 911 Chemistry Analyzer (Roche Diagnostics, Indianapolis, Ind.). Urinary PlGF determinations were performed with the PlGF ELISA (enzyme linked immunosorbent assay) kit (R&D Systems).

### Podocyturia

Random urine samples (25-50 mL each) were centrifuged for 8 minutes at 700 g at room temperature. The pellets were rinsed twice with human diploid fibroblast (HDF) solution. Next, the pellets were resuspended in Dulbecco's modified eagle's medium (DMEM) F-12 medium with 10% fetal bovine serum that was supplemented with antibiotics for the prevention of bacterial contamination. One-milliliter aliquots were plated in four-chamber, collagen-coated tissue culture slides, which was followed by overnight incubation at 37° C. in 5% CO₂. The next day, the media were removed, followed by two phosphate-buffered saline solution washes. Slides were fixed with 1 mL of ice cold methanol for 10 minutes at −20° C. Each of the four slide chambers was incubated with one of four different antibodies to podocyte proteins: podocalyxin (dilution, 1:40), podocin (dilution, 1:200), nephrin (dilution, 1:100), and synaptopodin (undiluted). After being washed with phosphate-buffered saline solution, a secondary fluorescein isothiocyanate-labeled antibody was added at a dilution of 1:40 for 30 minutes. The sediment was counterstained with Hoechst nuclear stain to facilitate differentiation of whole cells from cell fragments. Coverslips were mounted with Vectashield (Vector Labs, Burlington, Calif.), and the slides were viewed with a fluorescence microscope (Leica, Germany). Nucleated, positive-staining cells were considered to be podocytes. A renal pathologist, who was blinded to the clinical diagnosis and laboratory findings, evaluated each sample to determine the number of cells that were present and the percentage of cells that were stained for podocyte markers. Podocyturia was expressed as a ratio of the number of podocytes to the creatinine content of the respective urine sample, which was performed for each of the four podocyte markers.

### Statistical Methods

Descriptive statistics are reported for quantitative traits as means and SDs or as medians and interquartile ranges and for categoric traits as percentages. The operating characteristics of podocyte and angiogenic markers of preeclampsia were assessed by consideration of the trait either as a categoric measure (i.e., absence/presence) and estimation of its sensitivity and specificity or by the consideration of it as a quantitative measure and the generation of the receiver-operating characteristic (ROC) curve. The areas under the curve were estimated with confidence intervals and contrasted among markers with a method described elsewhere (DeLong et al., Biometrics, 44:837-45 (1988)). All statistical tests were carried out at the two-sided 0.05 significance level.

### Podocyturia

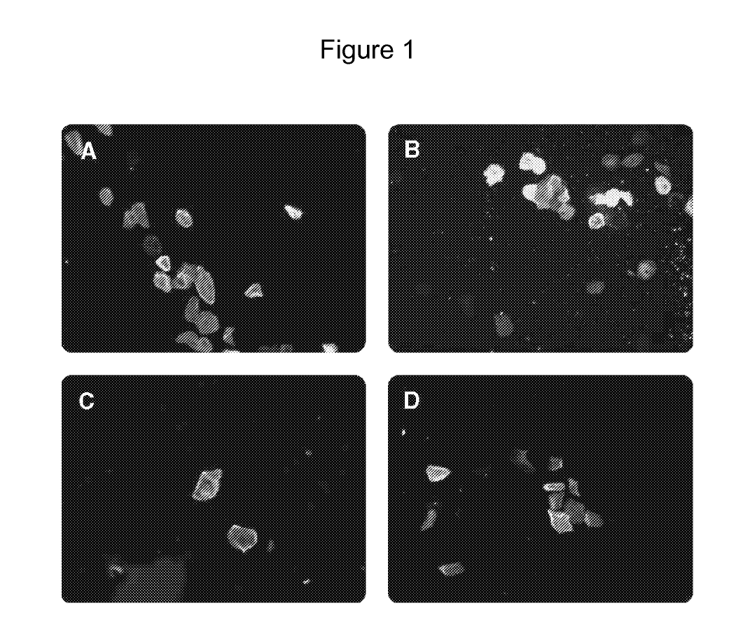

In the women with urinary measures of podocyturia (i.e., 15 cases and 16 control subjects), those women with preeclampsia or HELLP had podocin-positive cells in the urine (FIG. 1A), whereas none of the normotensive control subjects had any podocin-positive cells. Thus, the sensitivity and specificity of podocyturia, as determined by the podocin-positive cells, for the diagnosis of preeclampsia were both 100%. A positive correlation between the degree of proteinuria and podocyturia, as determined by podocin staining, was present (P=0.04).

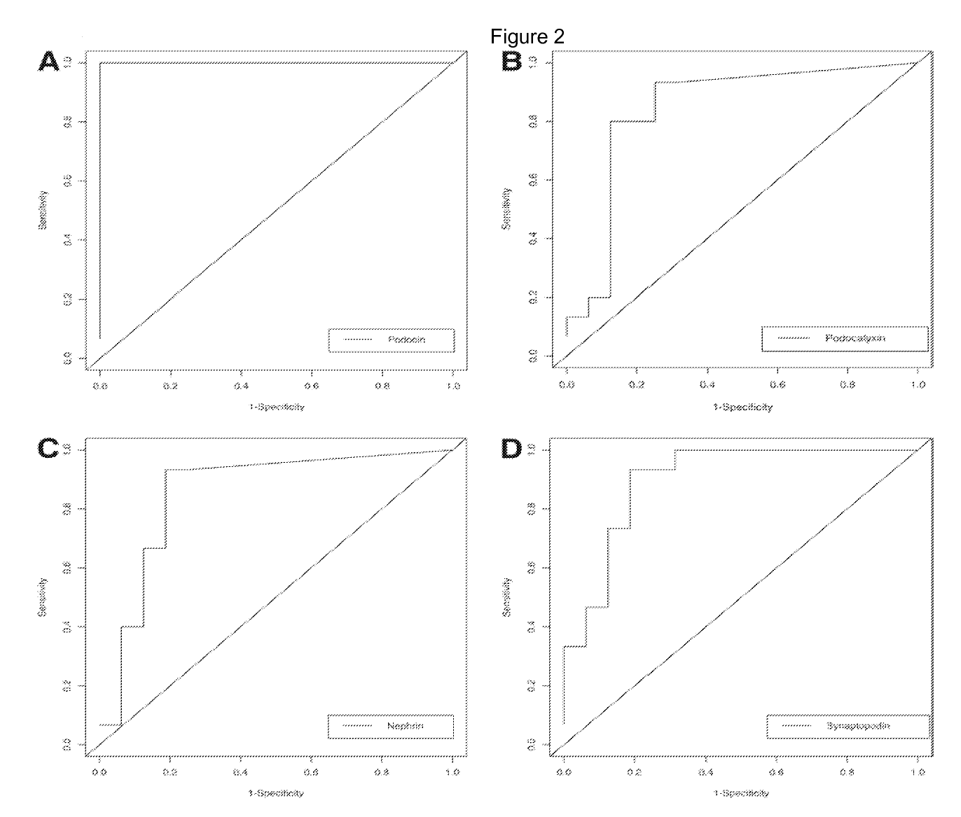

Compared with podocin, measurements of podocyturia that were based on podocalyxin, nephrin, and synaptopodin stains (FIGS. 1B, C, and D, respectively) had both lower sensitivity and specificity (Table 2). For sFLT-1, endoglin, and PlGF, the sensitivity and specificity were calculated twice, with two different cutoffs to define a positive test. The cutoffs for podocyturia (podocin, nephrin, podocalyxin, and synaptopodin) were expressed as cells per milligram of creatinine (Table 2). The ROC curves for all four podocyte markers were generated (FIG. 2), and their respective areas under the curve were compared as a measure of diagnostic accuracy.

TABLE 2
Test characteristics for markers of preeclampsia.

| | | | | | Pretest probability for preeclampsia | | | |
|---|---|---|---|---|---|---|---|---|
| | | | | | 5% | | 25% | |
| | | | | | Positive | Negative | Positive | Negative |
| | | | | | predictive | predictive | predictive | predictive |
| Test | Cutoff | | Sensitivity (%) | Specificity (%) | value (%) | value (%) | value (%) | value (%) |
| sFLT-1* | 7463 | pg/mL | 83 | 58 | 9.4 | 98.5 | 39.7 | 91.1 |
| | 9795 | pg/mL | 71 | 68 | 10.5 | 97.8 | 42.5 | 87.6 |
| Endoglin* | 21.3 | ng/mL | 94 | 58 | 10.5 | 99.5 | 42.7 | 96.7 |
| | 24.6 | ng/mL | 86 | 63 | 10.9 | 98.8 | 43.7 | 93.1 |
| Serum PlGF† | 84.92 | pg/mL | 74 | 58 | 8.5 | 97.7 | 37.0 | 87.0 |
| | 102.7 | pg/mL | 86 | 47 | 7.9 | 98.5 | 35.1 | 91.0 |
| Urine PlGF† | 1.22 | pg/mL‡ | 79 | 50 | 7.7 | 97.8 | 34.5 | 87.7 |
| | 2.18 | pg/mL† | 86 | 38 | 7.5 | 98.4 | 33.9 | 90.4 |
| Podocin* | 0.85 | cells† | 100 | 100 | 100.0 | 100.0 | 100.0 | 100.0 |
| Nephrin* | 0.75 | cells† | 93 | 75 | 16.4 | 99.5 | 55.4 | 97.0 |
| Podocalyxin* | 0.83 | cells† | 93 | 75 | 16.4 | 99.5 | 55.4 | 97.0 |
| Synaptopodin* | 1.11 | cells† | 93 | 81 | 20.5 | 99.5 | 62.0 | 97.2 |

*A positive test is defined as having a value higher than the cutoff.
†A positive test has a lower value than the cutoff.
‡Expressed per milligram of creatinine in the respective urine samples

The analysis indicated that podocin had a greater diagnostic accuracy than did podocalyxin (P=0.04) or nephrin (P=0.05) and possibly better than did synaptopodin (P=0.08); diagnostic accuracy of the other three markers (podocalyxin, nephrin, and synaptopodin) did not differ. For the cases, the rate of podocyte excretion, which was expressed as median cell number per milligram of creatinine, was 3.7 for podocin and synaptopodin, 5.0 for podocalyxin, and 3.3 for nephrin. For the control subjects, the rate of podocyte excretion for synaptopodin was 0.6, and 0 for podocalyxin, podocin, and nephrin. An additional control group consisted of women with gestational hypertension (n=6), essential hypertension (n=2), and preexisting proteinuria (n=3), who did not have clinical signs and symptoms of superimposed preeclampsia. None of these 11 women demonstrated podocyturia, as determined by podocin staining.

### Angiogenic Markers of Preeclampsia

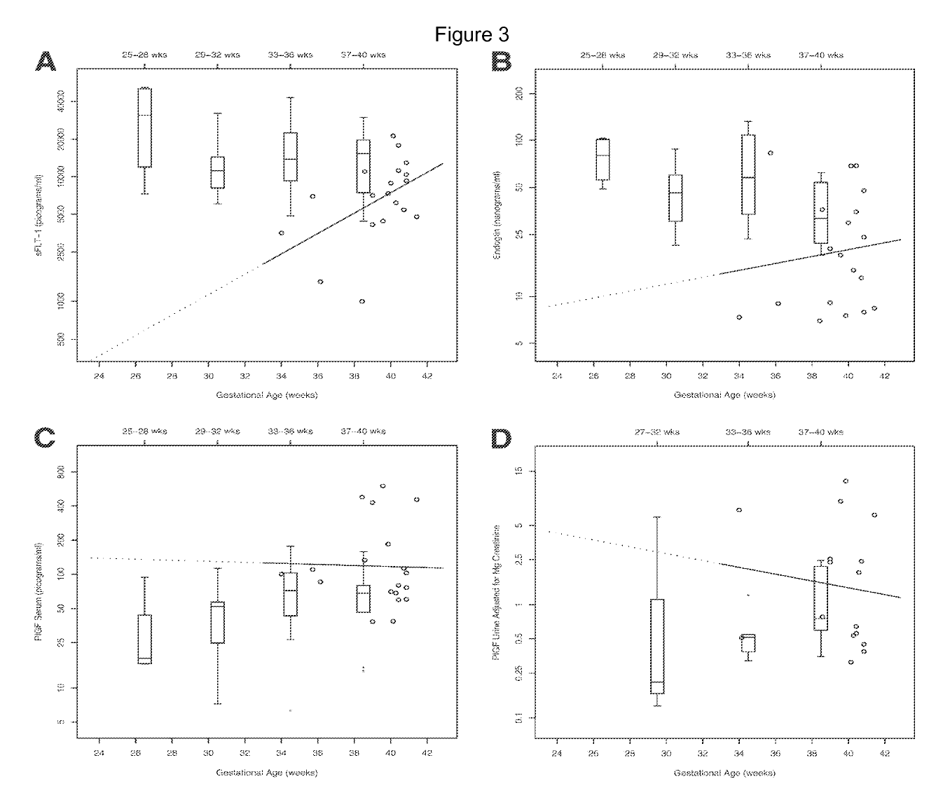

Serum sFlt-1 levels were significantly higher in women with preeclampsia or HELLP than in normotensive pregnant control subjects (17,326±12,124 pg/mL vs. 8,160±5,186 pg/mL; P<0.001; Table 3). Serum sFlt-1 levels did not differ significantly between patients with preeclampsia and HELLP (P=0.11). Patients with preeclampsia and HELLP displayed higher sFlt-1 levels than normal patients, if they delivered early in pregnancy (FIG. 3A). This difference became less apparent closer to full term delivery.

TABLE 3
Normal and preeclamptic levels of sFLT-1, endoglin, and PlGF.

| | | | | Preeclampsia + |
|---|---|---|---|---|
| Variable | Normal (n = 23) | Preeclampsia (n = 33) | HELLP (n = 11) | HELLP (n = 44) |
| sFLT-1 (pg/mL) | 8160 ± 5186 | 18,231 ± 11,216* | 14,711 ± 14,876* | 17,326 ± 12,124* |
| Endoglin (ng/mL) | 27.2 ± 23.9 | 56.5 ± 31.7* | 52.1 ± 32.7* | 55.4 ± 31.6* |
| Serum PlGF (pg/mL) | 173 ± 175 | 66.2 ± 44.2* | 59.8 ± 48.5* | 64.6 ± 44.7* |
| Urine PlGF (pg/mL | 2.94 ± 3.56 | 1.17 ± 1.54 | † | † |
| per mg creatinine) | | | | |

Data are given as mean ± SD.
*P < 0.05, compared with normal group.
† None of the 11 patients with HELLP had urine samples.

Serum soluble endoglin levels were significantly higher in women with preeclampsia or HELLP than in normotensive pregnant control subjects (55.4±31.6 ng/mL vs. 27.2±23.9 ng/mL; P<0.001). Serum soluble endoglin levels did not differ significantly between patients with preeclampsia and HELLP (P=0.69). The difference between normal and preeclamptic pregnancies was greater with an earlier delivery and became less apparent in those patients who were delivered at full term (FIG. 3B).

Serum-free PlGF levels were lower in women with preeclampsia or HELLP than in normotensive pregnant control subjects (64.6±44.7 pg/mL vs. 173±174.8 pg/mL; P=0.0005). Serum-free PlGF levels did not differ significantly between patients with preeclampsia and HELLP (P=0.36). In those patients delivering at an earlier gestational age, free PlGF levels were lower in patients with preeclampsia and HELLP vs. control subjects, but this difference became less apparent as pregnancies were carried towards full term (FIG. 3C).

There was a statistically insignificant trend towards lower urine PlGF levels in women with preeclampsia or HELLP, compared with normotensive pregnant control subjects (1.17±1.54 pg/mL/mg vs. 2.94±3.56 pg/mL/mg creatinine; P=0.11; Table 3). Urine PlGF levels in women with preeclampsia were not different than in normal women, regardless of gestational age at delivery (FIG. 3D).

### Angiogenic Factors as Diagnostic Tests for Preeclampsia: Comparison to Podocyturia

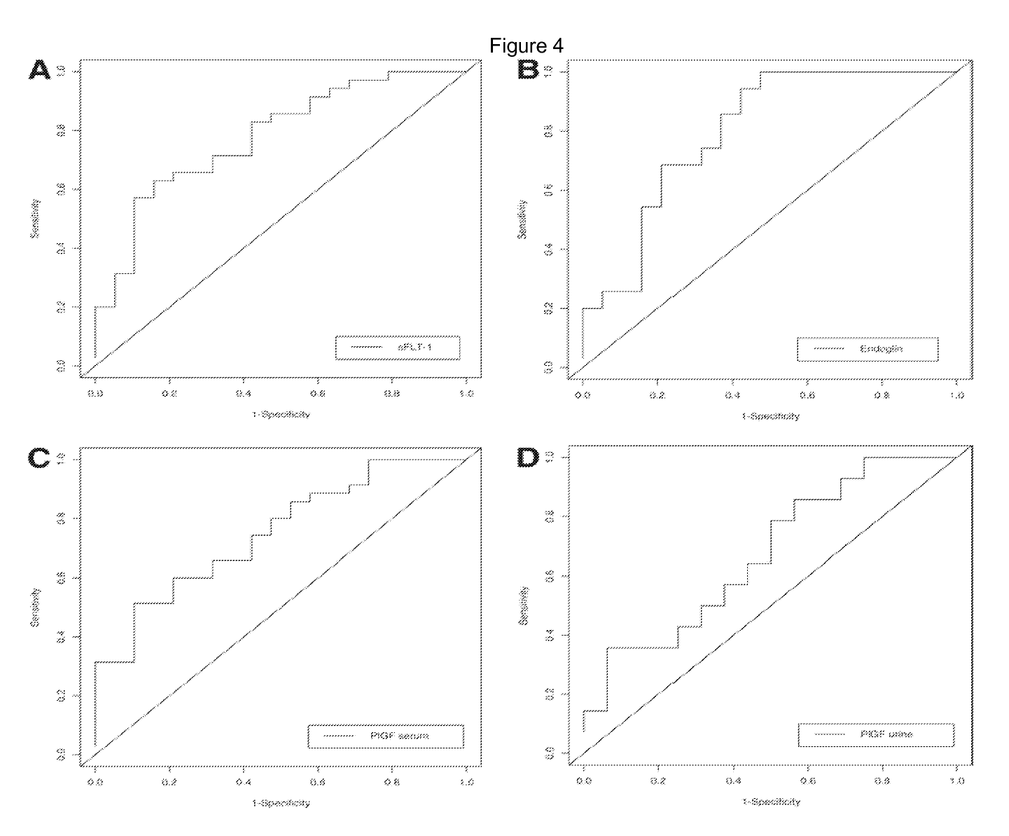

ROC curves were generated for sFlt-1, soluble endoglin, and both serum and urine PlGF (FIG. 4). The positive predictive value and the negative predictive value for podocyturia, as determined by the four podocyte-specific markers and the angiogenic factors that were evaluated (Table 2), were calculated. Because the value of a diagnostic test can depend on the pretest probability of disease, the diagnostic accuracy of each test was estimated for two different pretest probabilities: 5%, which reflects the pretest probability for preeclampsia in the general population, and 25%, which is a commonly cited percentage risk in women with preexisting hypertension. The negative predictive value did not differ between the podocyturia and angiogenic factor tests in patients with a low (5%) pretest probability. However, in patients with a pretest probability of 25%, the negative predictive value was higher with podocyturia. The positive predictive value was higher with podocyturia, compared with angiogenic factors tests in both the low and high pretest probability groups.

The results provided herein demonstrate that podocyturia (i.e., urinary excretion of podocytes) is present in patients with preeclampsia at the time of delivery. These cells retain the ability to attach to tissue culture plates in vitro, which indicates that they are viable. Urinary shedding of podocytes may contribute to proteinuria in preeclampsia, because these cells have a very limited regenerative capacity. Therefore, podocyturia may indicate podocyte loss from the glomerulus which may lead to a disruption of the glomerular filtration barrier and consequent proteinuria.

Podocyturia is present at the time of the clinical diagnosis of preeclampsia, and the number of podocytes can correlate with the degree of proteinuria. Urinary podocyte excretion, which was quantified by four podocyte-specific markers (namely, podocalyxin, podocin, nephrin, and synaptopodin), is a sensitive marker of renal damage and proteinuria in preeclampsia. Among these four markers, podocin exhibited a high sensitivity and specificity of 100% each. In addition, a positive correlation between the degree of proteinuria and podocyturia was found, which suggests a possible common/shared underlying pathogenic mechanism. Women with normotensive pregnancies and women with either hypertension or proteinuria, but in the absence of the clinical syndrome of preeclampsia, did not have podocyturia. Therefore, podocyturia does not appear to be merely a result of hypertensive kidney damage or a marker of proteinuria.

There are several possible explanations for podocin being a better marker for podocyturia in preeclampsia than podocalyxin, nephrin, or synaptopodin. Staining for podocalyxin, nephrin, and synaptopodin may be less specific, because these proteins, unlike podocin, are expressed in cells other than podocytes. These cells might be present in the urine particularly around the time of delivery, when urine samples can be contaminated with decidual, amniotic, and red and white blood cells. Moreover, staining for podocin may be more sensitive than staining for other podocyte proteins. Glomerular expression of nephrin and synaptopodin, but not podocin, was found to be decreased in kidney sections from women with preeclampsia. Consequently, podocytes that are shed in the urine may have lower expressions of nephrin and synaptopodin than podocin, which makes the latter a more sensitive marker of podocyte presence in the urine. It is particularly intriguing to postulate that podocyturia, as a marker of subclinical renal damage, may be detected before overt proteinuria and the full clinical picture of preeclampsia develops.

The results provided herein demonstrate that differences in sFlt-1, PlGF, and soluble endoglin levels between normotensive and preeclamptic pregnancies were greatest in those women who delivered earliest. Early delivery was a marker of severe disease that resulted in termination of pregnancy. These differences become less apparent as pregnancies are carried toward full term. There is a significant overlap in free PlGF and sFlt-1 values between mild forms of preeclampsia and normotensive pregnancies closer to full term, which could lead potentially to both false-positive and false-negative screening test results. In addition, no difference in urinary PlGF was observed between the cases (n=15) and control subjects (n=16), and no significant difference in circulating endoglin levels was observed between the cases of preeclampsia (n=33) and HELLP (n=11).

In summary, the results provided herein indicated that podocyturia is a marker of renal damage and proteinuria in preeclampsia.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing a pregnant mammal for preeclampsia, said method comprising determining whether or not a urine sample from said mammal contains an elevated level of urinary podocytes, wherein the presence of said elevated level indicates that said mammal has preeclampsia.

2. The method of claim 1, wherein said mammal is a human.

3. The method of claim 1, wherein said determining step comprises using an antibody to detect podocytes.

4. The method of claim 1, wherein said antibody is an anti-podocin antibody.

5. The method of claim 1, wherein said antibody is an anti-podocalyxin antibody.

6. The method of claim 1, wherein said antibody is an anti-nephrin antibody.

7. The method of claim 1, wherein said antibody is an anti-synaptopodin antibody.

8. The method of claim 1, wherein said method comprises classifying said mammal as having preeclampsia if said elevated level is present, and classifying said mammal as not having preeclampsia if said elevated level is not present.
